(19) United States
(12) Patent Application Publication
(10) Pub. No.: US 2011/0092819 A1
(43) Pub. Date: Apr. 21, 2011

(54) ULTRASONIC DIAGNOSIS APPARATUS AND ULTRASOINC DATA ACQUISITION METHOD

(75) Inventors: Masao TAKIMOTO, OTAWARA-SHI (JP); Muneki KATAGUCHI, Nasushiobara-shi (JP); Shigemitsu NAKAYA, Nasushiobara-shi (JP)

(73) Assignees: KABUSHIKI KAISHA TOSHIBA, TOKYO (JP); TOSHIBA MEDICAL SYSTEMS CORPORATION, OTAWARA-SHI (JP)

(21) Appl. No.: 12/901,709

(22) Filed: Oct. 11, 2010

(30) Foreign Application Priority Data
Oct. 21, 2009 (JP) 2009-242641

(51) Int. Cl. A61B 8/06

(52) U.S. Cl. 600/454

(57) ABSTRACT

According to one embodiment, an ultrasonic diagnosis apparatus includes an ultrasonic probe, a transmission/reception unit, an echo signal storage unit, a reception delay addition processing unit, a Doppler signal generating unit, and a focus position decision unit. The echo signal storage unit stores a plurality of echo signals obtained by the transmission/reception unit. The reception delay addition processing unit generates a plurality of reception signals corresponding to a plurality of reception focus positions by performing delay addition of the plurality of stored echo signals. The Doppler signal generating unit generates a plurality of Doppler signals corresponding to the plurality of reception focus positions from the generated reception signals. The focus position decision unit performs selection of a Doppler signal from the plurality of generated Doppler signals based on a blood flow characteristic and decides a reception focus position corresponding to the selected Doppler signal.

### CROSS-REFERENCE TO RELATED APPLICATIONS

This application is based upon and claims the benefit of priority from Japanese Patent Application No. 2009-242641, filed Oct. 21, 2009; the entire contents of which are incorporated herein by reference.

### FIELD

Embodiments described herein relate generally to an ultrasonic diagnosis apparatus and an ultrasonic data acquisition method.

### BACKGROUND

In general, when determining the severity of a patient's valvular heart disease, the user observes the motions of the entire heart and the motions of a valve in the B mode or the M mode, and then observes how the blood regurgitates due to the incomplete closure of the valve in the color mode. To check the degree of regurgitation, the user sets the focus position of ultrasonic waves on the regurgitating blood flow and observes the Doppler waveform being displayed then. Normally, the focus position of ultrasonic waves is set near a valve port. The user then determines the severity of the patient's valvular disease by use of the measuring function, i.e., by measuring the maximum flow velocity of regurgitating blood flow based on an ultrasonic Doppler signal obtained at the set focus position and by measuring the volume of regurgitating blood flow. The maximum flow velocity of regurgitating blood and the volume of regurgitating blood are important information for determining the severity of the patient's valvular heart disease.

However, if the valvular heart disease is severe, there may be a case where the flow velocity of the regurgitating blood is very high near the valve port, and the rate of regurgitating blood volume is very large. In this case, the blood regurgitates markedly near the valve port, and the direction of regurgitation is not constant. It should be noted that data (Doppler signals) based on which measurement is made is dependent on the sensitivity of the apparatus, the signal-to-noise ratio, and the scan technique of the user. For this reason, even when the user sets a focus position near the valve port at which blood is regurgitating, an obtained Doppler signal does not always indicate the maximum flow velocity of regurgitating blood and the maximum rate of regurgitating blood volume. Under these circumstances, the Doppler signal measured at the focus position near the valve port in the prior art may not allow accurate measurement of the maximum flow velocity of regurgitating blood and the maximum rate of regurgitating blood volume.

### BRIEF DESCRIPTION OF THE DRAWINGS

FIG. 1 is a block diagram showing the arrangement of an ultrasonic diagnosis apparatus 10 according to this embodiment;

FIG. 2 is a flowchart showing an example of a processing procedure in a focus position adjustment function according to this embodiment;

FIG. 3 is a view showing an example in which no focus mark indicating a reception focus position is set on a sound ray marker for a continuous wave Doppler mode on the image generated in the B mode and the color Doppler mode according to this embodiment;

FIG. 4 is a graph showing the trace image of a Doppler waveform obtained by an FFT computation unit 242 according to this embodiment and an sMAP corresponding to the trace image;

FIG. 5 is a graph showing the trace image of a Doppler waveform obtained by the FFT computation unit 242 at a reception focus position different from that in FIG. 4 and an sMAP corresponding to the trace image;

FIG. 6 is a graph showing an example of the relationship between reception focus positions and the maximum blood flow velocities at the respective reception focus positions according to this embodiment;

FIG. 7 is an example of a graph showing a focus mark indicating a reception focus position R corresponding to the maximum blood flow velocity in FIG. 6 on the image generated by the B mode and the color Doppler mode according to this embodiment, and the trace image of a Doppler waveform obtained by the FFT computation unit 242 at the reception focus position R;

FIG. 8 is a graph showing the angles in the scanning direction and the average values of the amplitudes of ultrasonic beams, with the sound ray marker being set to 0°, according to this embodiment;

FIG. 9 is a graph showing an example of the relationship between reception focus positions and areas (blood flow volumes) surrounded by the trace images of Doppler waveforms at the respective reception focus positions according to this embodiment;

FIG. 10 is a view showing an example of the trace images of Doppler waveforms obtained by the FFT computation unit 242 from a plurality of Doppler signals in one heartbeat period, at different reception focus positions, in synchronism with electrocardiogram (ECG) signals according to this embodiment;

FIG. 11 is a view showing an example of the trace image of a Doppler waveform obtained by the FFT computation unit 242 upon setting a reception focus position near the valve port in the prior art; and

FIG. 12 is a block diagram showing the arrangement of a conventional ultrasonic diagnosis apparatus 100.

### DETAILED DESCRIPTION

In general, according to one embodiment, an ultrasonic diagnosis apparatus includes an ultrasonic probe, a transmission/reception unit, an echo signal storage unit, a reception delay addition processing unit, a Doppler signal generating unit, and a focus position decision unit. The transmission/reception unit transmits and receives ultrasonic waves to and from an object through the ultrasonic probe. The echo signal storage unit stores a plurality of echo signals obtained by the transmission/reception unit. The reception delay addition processing unit generates a plurality of reception signals corresponding to a plurality of reception focus positions upon delay addition of a plurality of stored echo signals. The Doppler signal generating unit generates a plurality of Doppler signals corresponding to a plurality of reception focus positions from the generated reception signals. The focus position decision unit selects a Doppler signal from a plurality of generated Doppler signals based on blood flow characteristics, and decides a reception focus position corresponding to the selected Doppler signal.

An embodiment will be described below with reference to the views of the accompanying drawing.

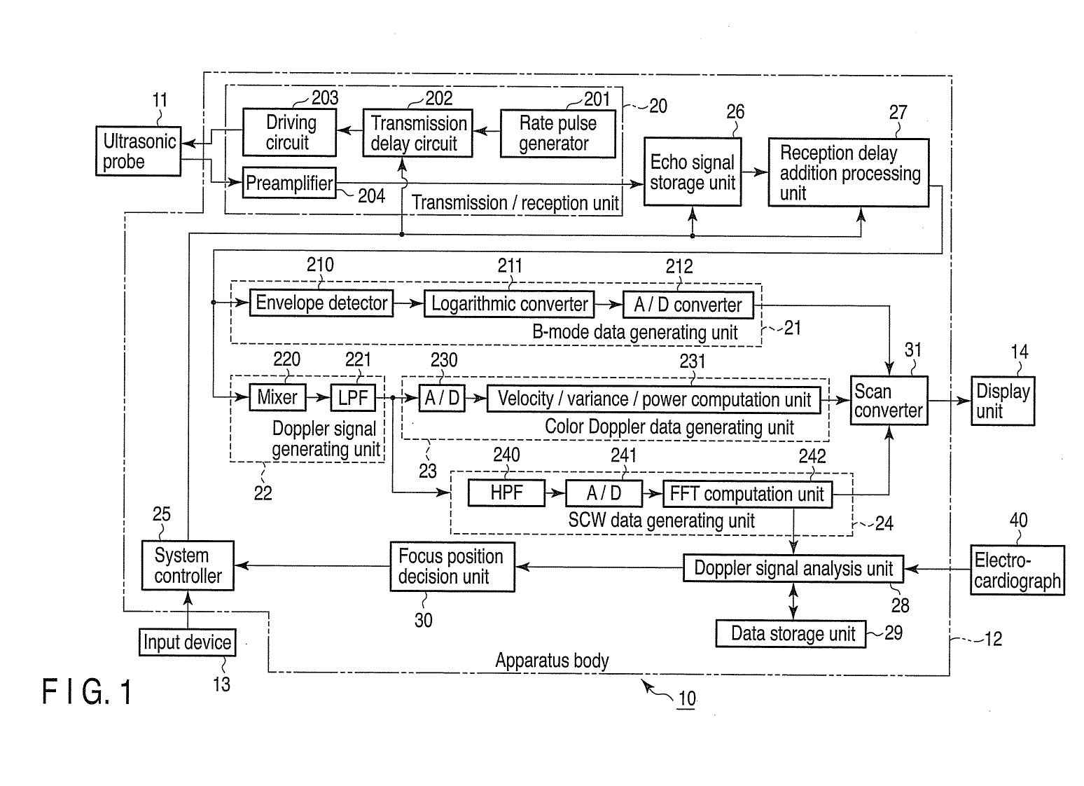

FIG. 1 is a block diagram showing the arrangement of an ultrasonic diagnosis apparatus 10 according to this embodiment. As shown in FIG. 1, the ultrasonic diagnosis apparatus 10 includes an ultrasonic probe 11, an apparatus body 12, an input device 13 which is connected to the apparatus body 12 to input various commands, instructions, and information from the user to the apparatus body 12, and a display unit 14. The input device 13 includes a trackball, switch buttons, a mouse, and a keyboard (none of which are shown) which are used to, for example, set a region of interest (ROI). The apparatus body 12 also includes a transmission/reception unit 20, a B-mode data generating unit 21, a Doppler signal generating unit 22, a color Doppler data generating unit 23, a continuous wave Doppler (to be referred to as SCW hereinafter) data generating unit 24, a system controller 25, an echo signal storage unit 26, a reception delay addition processing unit 27, a Doppler signal analysis unit 28, a data storage unit 29, a focus position decision unit 30, and a scan converter 31. In addition, the apparatus body 12 is separately provided with an electrocardiograph 40 to acquire electrocardiographic waves of an object.

The ultrasonic probe 11 includes piezoelectric transducers as acousto-electric reversible transducers such as piezoelectric ceramic transducers. A plurality of piezoelectric transducers are arranged in parallel and mounted on the distal end of the ultrasonic probe 11. Note that one transducer forms one channel.

The transmission/reception unit 20 includes a rate pulse generator 201, a transmission delay circuit 202, a driving circuit 203, and a preamplifier 204. The rate pulse generator 201 decides the cycle period of transmission ultrasonic waves based on the reference signal (clock) supplied from a reference signal generating unit (not shown), and generates rate pulses in the B mode method, the color Doppler method, and the pulse Doppler method.

The transmission delay circuit 202 gives the rate pulses or continuous waves supplied from the rate pulse generator 201 delay times necessary to focus ultrasonic waves into a beam and decide transmission directivity. The driving circuit 203 generates pulse-like driving signals for driving the piezoelectric transducers built in the ultrasonic probe 11 in synchronism with rate pulses. In the continuous wave Doppler mode, the driving circuit 203 continuously generates driving signals.

The piezoelectric transducers convert echoes from the object into a plurality of electrical signals (echo signals). The preamplifier 204 amplifies each of a plurality of echo signals and converts it into a digital signal.

The echo signal storage unit 26 stores echo signals output from the transmission/reception unit 20 before reception delay addition. The reception delay addition processing unit 27 also called a digital beam former gives an echo signal from each piezoelectric transducer, stored in the echo signal storage unit 26, a delay time necessary to focus a reception ultrasonic wave from a predetermined depth so as to obtain a small beam width and a delay time necessary to set strong reception directivity for a reception ultrasonic wave from a predetermined direction. A set of delay times for a plurality of piezoelectric transducers will be referred to as a reception delay pattern. The system controller 25 stores a plurality of reception delay patterns corresponding to different focal depths in an internal memory (not shown). The reception delay addition processing unit 27 adds a plurality of echo signals in accordance with a reception delay pattern from the system controller 25. This addition will enhance a reflection component from a direction corresponding to reception directivity. This transmission directivity and reception directivity decide the total directivity of ultrasonic transmission/reception (this directivity decides so-called “ultrasonic scanning lines”).

The B-mode data generating unit 21 includes an envelope detector 210, a logarithmic converter 211, and an analog/digital converter (to be referred to as an A/D convert hereinafter) 212. The envelope detector 210 performs envelope detection of an input signal to the B-mode data generating unit 21, i.e., the reception signal output from the reception delay addition processing unit 27. The logarithmic converter 211 relatively enhances weak signals by logarithmically converting the amplitudes of detected signals. The A/D convert 212 converts an output signal from the logarithmic converter 211 into a digital signal and generates B-mode data.

The Doppler signal generating unit 22 includes a mixer 220 and a low-pass filter (to be referred to as an LPF hereinafter) 221. The mixer 220 mixes an output signal from the reception delay addition processing unit 27 with a reference signal having a frequency f₀ equal to that of a transmission frequency. This mixing allows to obtain a signal having a component with a Doppler shift frequency f_{d} and a frequency component (2f₀+f_{d}). The LPF 221 removes the signal with the high frequency component (2f₀+f_{d}) from the two kinds of frequency components from the mixer 220. By removing the signal with the high frequency component (2f₀+f_{d}), the Doppler signal generating unit 22 generates a Doppler signal having the component with the Doppler shift frequency f_{d}. Note that the Doppler signal generating unit 22 may use a quadrature detection scheme.

The color Doppler data generating unit 23 includes a 2-channel A/D converter 230 and a velocity/variance/power computation unit 231. The A/D converter 230 converts the Doppler signal output from the LPF 221 of the Doppler signal generating unit 22 or the quadrature-detected analog signal into a digital signal. Although not shown, the velocity/variance/power computation unit 231 includes an MTI filter and an auto-correlation computation unit. The MTI filter removes Doppler components (clutter components) caused by the respiratory movement or pulsatory movement of an organ from the Doppler signal output from the A/D converter 230. The auto-correlation computation unit calculates the auto-correlation value of the Doppler signal obtained by extracting only blood flow velocity information by the MTI filter, and further calculates the average velocity, variance, and the like of the blood flow based on the auto-correlation value. The color Doppler data generating unit 23 generates color Doppler data from the average velocity, variance, and the like of the blood flow based on a plurality of Doppler signals.

The SOW data generating unit 24 includes a high-pass filter (to be referred to as an HPF hereinafter) 240, an analog/digital converter (to be referred to as an A/D converter hereinafter) 241, and a fast Fourier transform (to be referred to as FFT hereinafter) computation unit 242. The HPF 240 removes Doppler components (clutter components) caused by the respiratory movement or pulsatory movement of an organ from the Doppler signal generated by the Doppler signal generating unit 22. The A/D converter 241 converts the Doppler signal output from the HPF 240 into a digital signal. The FFT computation unit 242 frequency-analyzes the Doppler signal converted into the digital signal by the A/D converter 241 by fast Fourier transform. The SCW data generating unit 24 generates SCW data from frequency analysis based on a plurality of Doppler signals. The display unit 14 displays the SCW data as shown on the left side of FIG. 4. The ordinate represents velocity. The abscissa indicates the time when SCW data was obtained. The abscissa expressed as Velocity represents zero velocity.

The Doppler signal analysis unit 28 generates one sMAP for a trace image of the Doppler waveform based on the frequency analysis by the FFT computation unit 242. That is, an sMAP is generated for each reception focus position. An sMAP is a chart obtained by searching the above trace image for an arbitrary period (analysis period) for each frequency in the time axis direction, assigning “1” to a frequency band, i.e., a velocity band, in which Doppler signals (velocity components) equal to or more than a specific threshold exist, and assigning “0” to a frequency band, i.e., a velocity band, in which no signals exist. That is, an sMAP is a chart indicating the presence/absence of blood flow components. For example, the right side of FIG. 4 shows an sMAP corresponding to the trace image of the Doppler waveform shown on the left side of FIG. 4. In this case, the interval between A and B corresponds to the signal-present area of the trace image. The maximum value of blood flow velocity (regurgitation in this case) (to be referred to as the maximum flow velocity hereinafter) is obtained from the generated sMAP. For example, in the sMAP on the right side of FIG. 4, the point corresponding to B indicates the maximum flow velocity (a negative velocity component because of regurgitation). Note that it is possible to obtain a maximum flow velocity by generating an sMAP from the absolute values of velocity components, e.g., the absolute values of velocities in the trace image of the Doppler waveform on the left side of FIG. 4. A maximum flow velocity may be obtained by generating an sMAP from the regurgitation components of the blood flow velocities, e.g., the velocity band equal to or less than the abscissa expressed as Velocity on the left side of FIG. 4. When generating an sMAP, the user selectively issues an instruction to use the trace image of the Doppler waveform without any change, to use the absolute values of the trace image, or to use only the regurgitation components of the trace image.

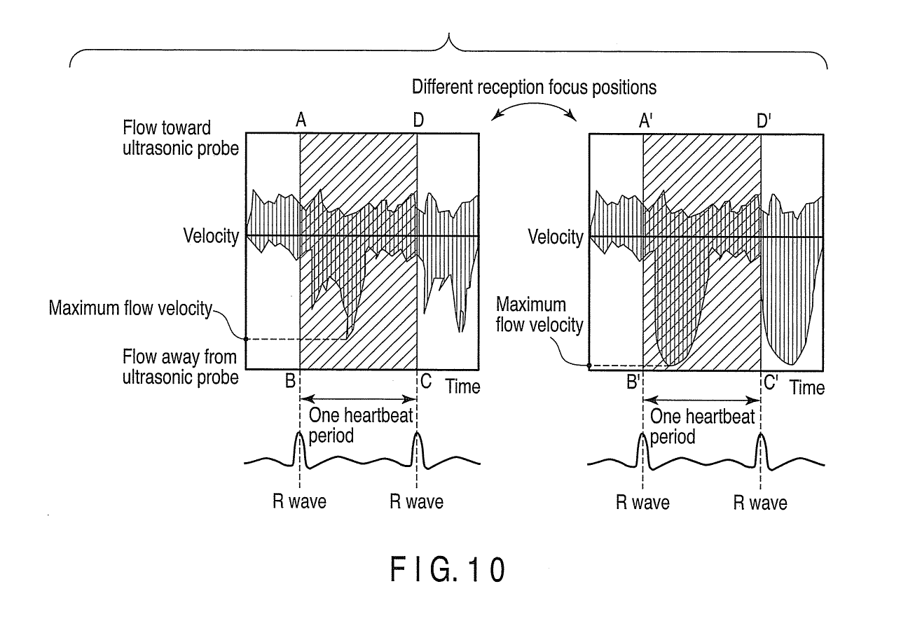

The arbitrary period can be set to, for example, one heartbeat period, in synchronism with the electrocardiograph 40 (to be described later). For example, FIG. 10 is a view showing the trace images of Doppler waveforms obtained at different reception focus positions, each of which is obtained by the FFT computation unit 242 from a plurality of Doppler signals in one heartbeat period. Referring to FIG. 10, this apparatus generates one sMAP for the trace image of the Doppler waveform inside a rectangle ABCD in one heartbeat period, and another sMAP for the trace image of the Doppler waveform inside a rectangle A′B′C′D′. The apparatus then obtains maximum flow velocities from the respective sMAPs. Note that the Doppler signal analysis unit 28 can also obtain the area of the regurgitation range surrounded by the trace image of the Doppler waveform. This area corresponds to the flow rate of blood flowing near the reception focus. Note that a maximum flow velocity, the absolute value of a velocity component, the maximum flow velocity based on the regurgitant blood flow velocity, the maximum blood flow rate, and the like are called blood flow characteristics.

The data storage unit 29 stores the maximum flow velocity obtained by the Doppler signal analysis unit 28 and a reception focus position corresponding to the maximum flow velocity. Note that the data storage unit 29 can also store the area surrounded by the above trace image and a reception focus position corresponding to the area.

Based on a plurality of maximum flow velocities stored in the data storage unit 29 and reception focus positions corresponding to the respective maximum flow velocities, the focus position decision unit 30 decides a reception focus position corresponding to a Doppler signal including the highest velocity of the maximum flow velocities. Note that based on the areas surrounded by the plurality of trace images stored in the data storage unit 29 and reception focus positions corresponding to the respective areas surrounded by the trace images, the focus position decision unit 30 can also decide a reception focus position corresponding to a Doppler signal having the largest value of the areas. The user selectively issues an instruction to specify a reception focus position based on a maximum flow velocity or to specify a reception focus position based on a blood flow rate.

The system controller 25 reads out transmission/reception conditions and apparatus control programs from the internal memory based on the mode, ROI, pattern list, and transmission start/end instructions selected or set by the user via the input device 13, and controls the ultrasonic diagnosis apparatus 10 in accordance with them. The apparatus also sets, on an image displayed on the display unit, a sound ray marker for the execution of the continuous wave Doppler method which is input by the user via the input device 13.

The scan converter 31 converts the B-mode data, color Doppler data, and SCW data respectively generated, in the unit of the scanning direction, by the B-mode data generating unit 21, the color Doppler data generating unit 23, and the SCW data generating unit 24, into scanning line signal strings in a general video format typified by a TV format, thereby generating ultrasonic diagnosis images as display images.

The display unit 14 displays morphological information and blood information in the living body as images based on video signals from the scan converter 31. The display unit 14 displays the sound ray marker and focus mark set by the system controller 25 on an image formed from B-mode data and an image formed from color Doppler data.

The electrocardiograph (ECG) 40 obtains a graph recording temporal changes due to the electric phenomenon of the heart of the object, i.e., an electrocardiogram (ECG waveform), by measurement. The electrocardiographic waveform signal detected by the electrocardiograph 40 is stored in the internal memory and is sent to the display unit 14 to be displayed as an electrocardiographic waveform, as needed.

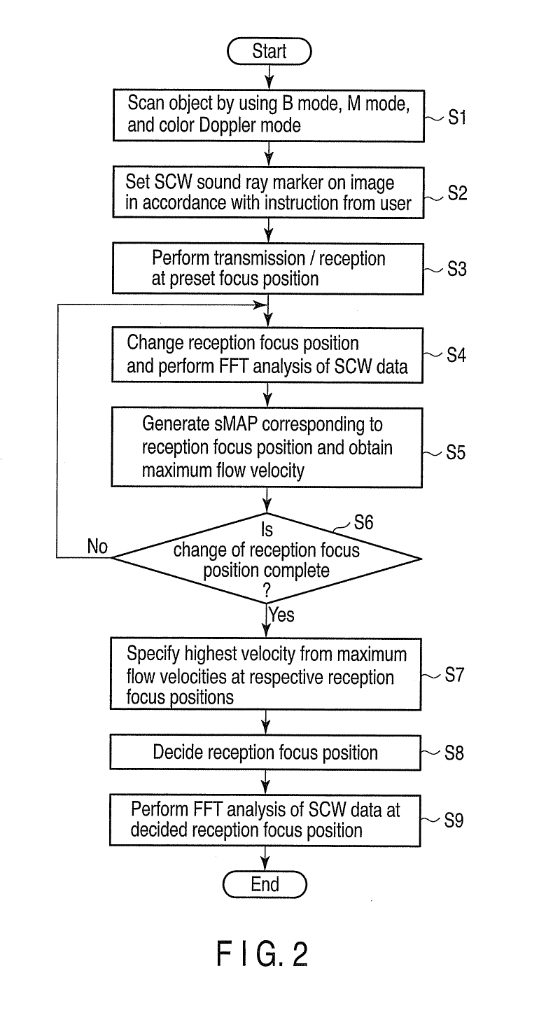

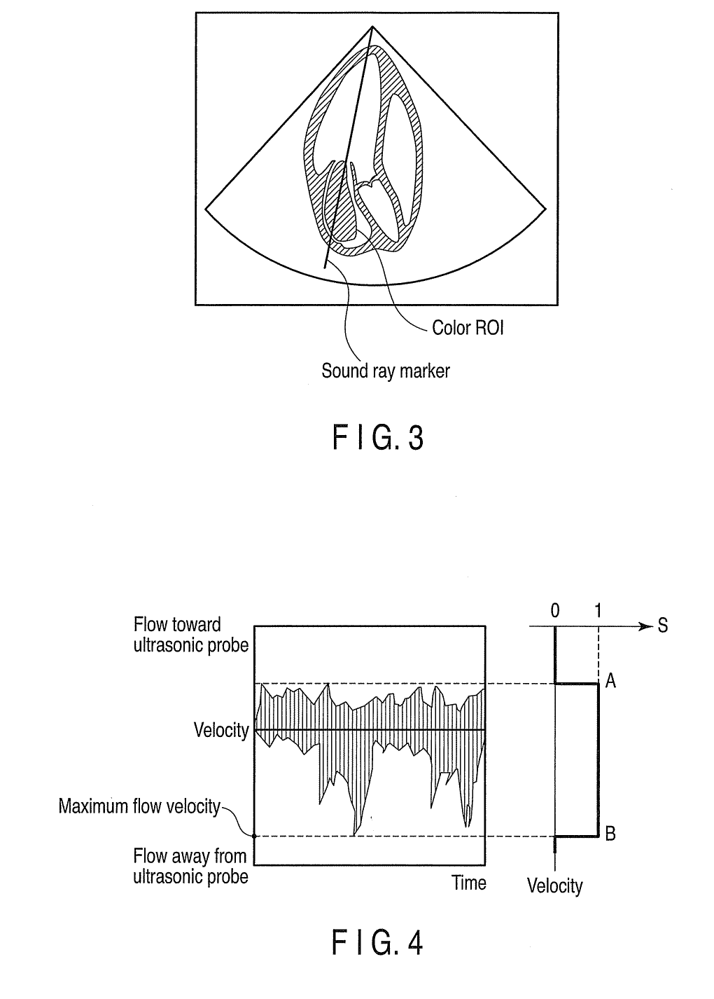

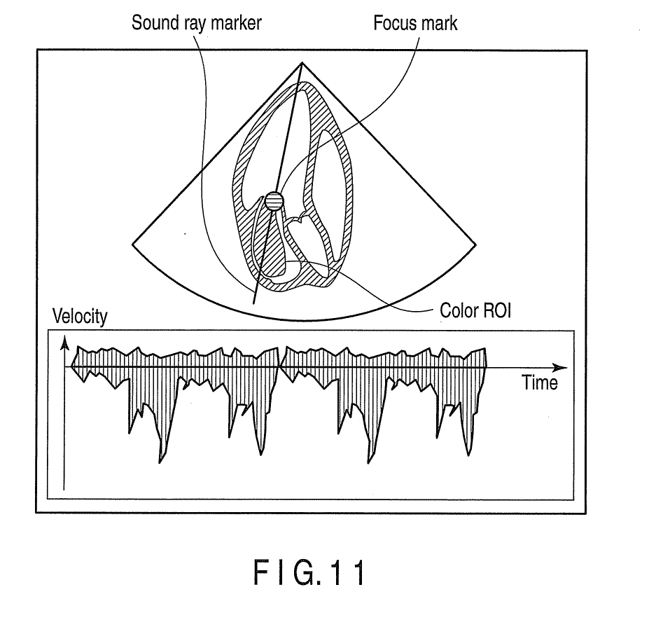

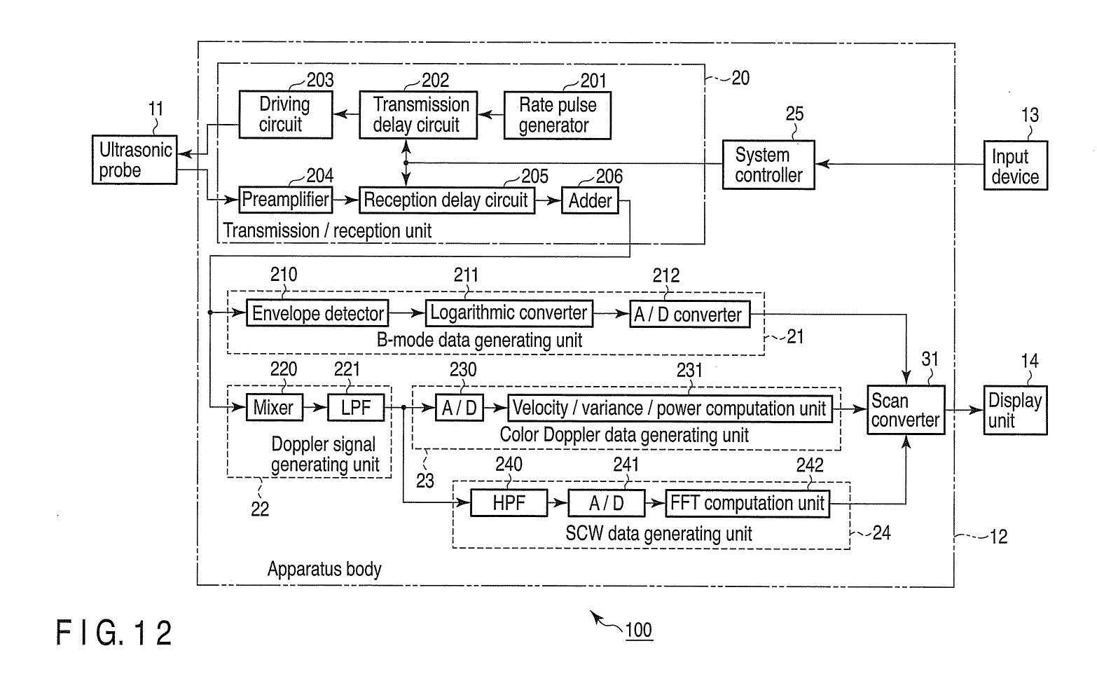

The operation of the focus position adjustment function of the ultrasonic diagnosis apparatus 10 will be described next with reference to the flowchart shown in FIG. 2. First of all, this function scans a region including the heart of an object by using the B mode under the control of the system controller 25 (step S1). The display unit 14 displays the scanned image. The function superimposes, on the displayed image, a sound ray marker for the execution of the continuous wave Doppler method under the control of the system controller 25 in accordance with an instruction to decide continuous wave Doppler conditions from the user via the input device 13 (step S2). FIG. 3 is a view showing how a sound ray marker is set on the color Doppler mode displayed on the display unit 14. According to the prior art, as shown in FIG. 11, the user sets a reception focus mark on a sound ray marker. This embodiment can automatically optimize a reception focus position without making the user operate a reception focus mark. This obviates the need to display a reception focus mark on the sound ray marker in FIG. 3. Note that like the conventional ultrasonic diagnosis apparatus 100, this apparatus can display a reception focus mark on a sound ray marker.

The transmission/reception unit 20 transmits and receives continuous ultrasonic waves throughout at least one heartbeat period to perform the continuous wave Doppler method at a preset reception focus position on the above sound ray (step S3). In this case, the focus position during scanning is, for example, a position corresponding to an intermediate depth of field along a sound ray. Note that a focus position at which transmission and reception are performed may be a position corresponding to half the length of a color ROI (region of interest) along a sound ray. The user can set a focus position, at which transmission and reception are performed, to an arbitrary depth in advance. It is possible to set, as a focus position at which transmission and reception are performed, the position of a reception focus mark which is displayed on a sound ray marker. The user can select each focus position, at which the above transmission and reception are performed, via the input device 13. The echo signal storage unit 26 stores echo signals obtained before reception delay processing by transmission/reception of ultrasonic waves.

Based on the depth of field and the width intervals of reception focus positions, a plurality of reception focus positions are determined. If a maximum flow velocity is detected at reception focus positions, those reception focus positions are changed to other reception focus positions at which the maximum flow velocity is not detected. The system controller 25 supplies the reception delay addition processing unit 27 with reception delay patterns corresponding to the reception focus positions where the maximum flow velocity is not detected (i.e., the reception focus positions after the change). The reception delay addition processing unit 27 performs reception delay addition processing for all the echo signals in one heartbeat period in accordance with each reception delay pattern. With this operation, the reception delay addition processing unit 27 generates a plurality of reception signals in different cardiac phases corresponding to the same reception delay pattern (the same reception focus position). The system controller 25 performs FFT processing for each of the plurality of generated reception signals (step S4). This generates a plurality of Doppler signals in different cardiac phases. The system controller 25 generates a Doppler waveform from the plurality of Doppler signals in the different cardiac phases. The system controller 25 generates an sMAP corresponding to the generated Doppler waveform and obtains a maximum flow velocity (step S5). The data storage unit 29 stores the obtained maximum flow velocity and a reception focus position (reception delay pattern) corresponding to the maximum flow velocity. Note that in step S5, it is possible to obtain the area of the regurgitation range surrounded by the trace image of a Doppler waveform corresponding to the volume of blood flowing near the reception focus. In this case, the data storage unit 29 stores the area of the regurgitation range and a reception focus position (reception delay pattern) corresponding to the area.

The operations in steps S4 and S5 are repeated until corresponding maximum flow velocities are detected at the reception focus positions, i.e., until the reception focus positions are successfully changed to new positions (step S6). With the above procedure, the system controller 25 generates a plurality of Doppler waveforms at different reception focal depths (reception focus positions).

The above repetitive operation in steps S4 to S6 will be described below.

It is possible to change a reception focus position (reception delay pattern) an arbitrary width, e.g., 2 cm, 1 cm, or 0.5 cm, at a time. In this case, reception delay patterns corresponding to these widths are supplied to the reception delay addition processing unit 27 to perform the processing in step S4, thereby generating Doppler waveforms at desired reception focus positions. The smaller the width of change, the larger the number of times of processing in steps S4 and S5, and hence the more the time required. However, it is possible to obtain more accurate maximum flow verocities. The completion of the change of the reception focus position in step S6 in FIG. 2 means that if, for example, the depth of field is 14 cm and the reception focus position is changed at equal width intervals of 1 cm, the reception focus position (reception delay pattern) is changed 14 times as the reception delay is changed from 0 cm to 14 cm. This operation obtains the relationship between 15 combinations of reception focus positions and maximum flow velocities. Note that applying the areas of regurgitation ranges surrounded by the trace images of Doppler waveforms corresponding to the volumes of blood flowing near reception focuses and the corresponding reception focus positions to the above case will obtain the relationship between 15 combinations of reception focus positions and the areas of regurgitation ranges.

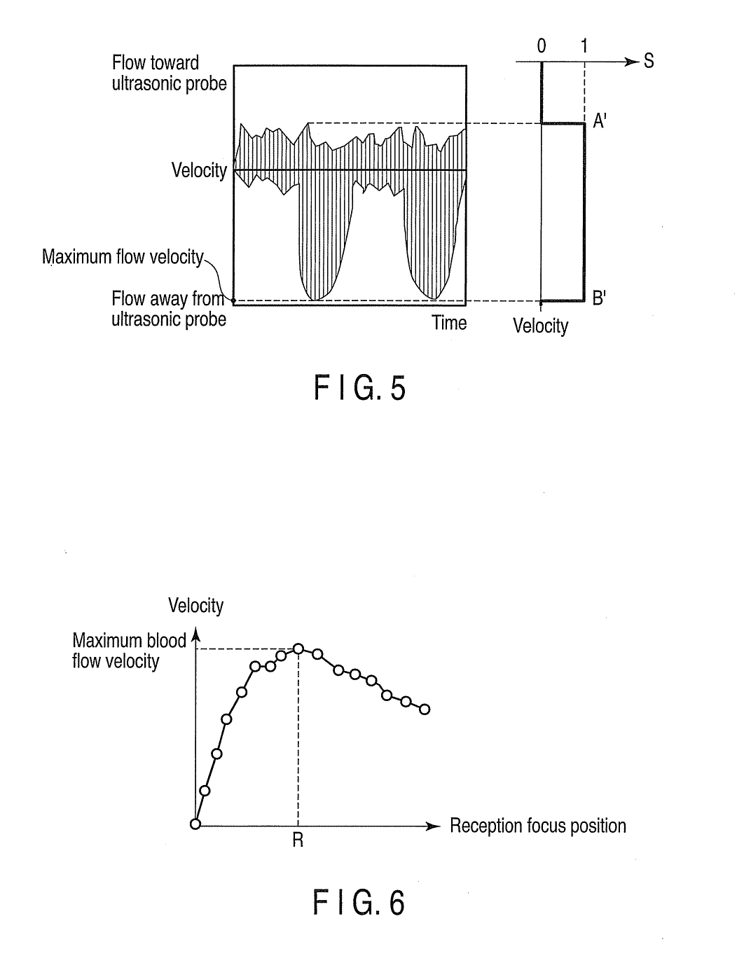

Changing a reception focus position may obtain, for example, the trace image of a Doppler waveform and the sMAP shown in FIG. 5. A maximum flow velocity B′ in FIG. 5 is higher than a maximum flow velocity B in FIG. 4. The envelope of the Doppler waveform in FIG. 5 is easier to trace than the Doppler waveform in FIG. 4. FIG. 6 is an example of a graph showing the relationship between a plurality of maximum flow velocities stored in the data storage unit 29 and reception focus positions corresponding to the respective maximum flow velocities. The focus position decision unit 30 specifies the highest velocity of a plurality of maximum flow velocities corresponding to a plurality of reception focus positions based on the graph of FIG. 6 (step S7). Note that it is possible to obtain the highest velocity of maximum flow velocities without generating the graph of FIG. 6.

FIG. 9 is an example of a graph showing the relationship between the areas of a plurality of regurgitation ranges and reception focus positions corresponding to the respective areas in a case in which the reception focus position is changed. It is also possible to obtain the largest area (the maximum flow rate of regurgitating blood) based on the graph of FIG. 9 in step S7.

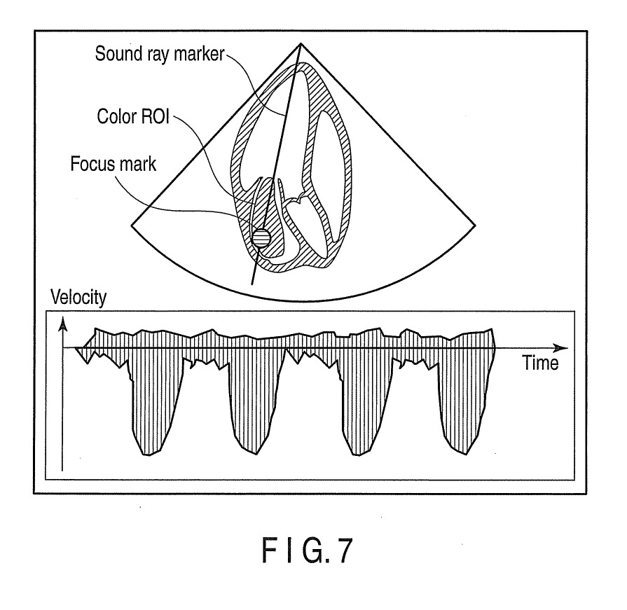

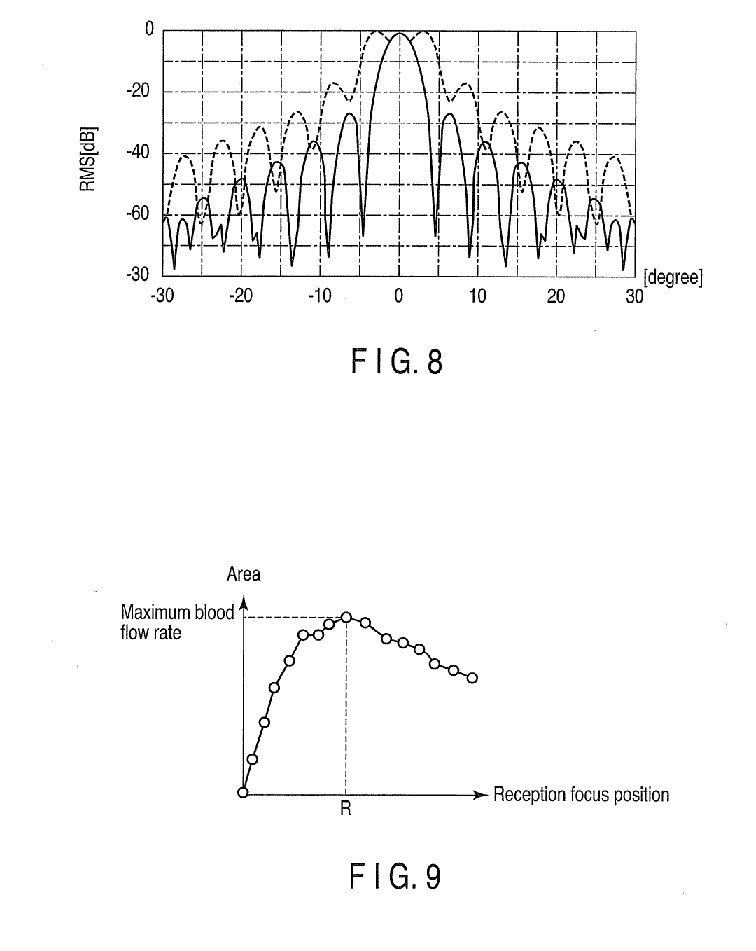

This apparatus decides a reception focus position R corresponding to the maximum flow rate or the maximum area obtained in step S7 based on the graph of FIG. 6 or 9 (step S8). FIG. 7 is a view showing a Doppler waveform at a reception focus position corresponding to the highest velocity of maximum flow velocities, and a sound ray marker and a focus mark at the position R which are superimposed on the color Doppler mode. As shown in FIG. 7, in some cases, the trace image of a Doppler waveform is made to appear clearly by moving a reception focus position to a position away from the valve port instead of setting the reception focus position at a regurgitation position (the focus mark in FIG. 11) near the valve port from which blood is regurgitating. Such situation occurs especially when the direction of regurgitation changes (fluctuates) within one heartbeat period instead of blood regurgitating in a predetermined direction. FIG. 8 is a graph showing angles in the scanning direction and the average values of the amplitudes of ultrasonic beams, with a sound ray marker being set at 0°. The solid line in FIG. 8 indicates an example of the ultrasonic beam profile in FIG. 11 when a reception focus is decided on regurgitation near the valve port. The broken line in FIG. 8 indicates an ultrasonic beam profile in FIG. 7 when a reception focus is decided at a position (a position corresponding to a deep focal depth) away from the valve port. The broken line indicates deeper focal depths than the solid line, and hence exhibits a larger beam width of a main beam near 0°. Increasing the beam width in this manner brings a better effect in the case of regurgitation with fluctuations in blood flow velocity.

Referring back to the flowchart of FIG. 2, the focus position decision unit 30 decides a reception focus position (reception delay pattern) (step S8). The system controller 25 then performs reception delay corresponding to the decided reception focus position for an echo signal again before reception delay processing, and performs FFT analysis of the obtained SCW data (step S9). Note that it is possible to store the results of FFT analysis of SCW data (step S4) in the internal memory in advance and read out the result of FFT analysis of SCW data (step S4) corresponding to the reception focus position decided by the focus position decision unit 30. The focus position decision unit 30 may perform scanning in step S1 as pre-scanning to decide a reception focus position. The system controller 25 then may execute main scanning upon matching a transmission focus position with the decided reception focus position, and perform FFT analysis of obtained SCW data. The user selectively issues an instruction to perform reception delay again from an echo signal at the decided reception focus position, to read out the result of FFT analysis stored in advance, or to perform re-scanning upon matching of a transmission focus position.

As described above, an optimal reception focus position showing a maximal flow velocity is automatically determined by looking at how the blood characteristics vary in accordance with the gradual movement of the reception focus position. The user does not have to be conscious of the focus position, and the apparatus automatically detects the optimal focus position. The Doppler waveform measured at that focus position enables accurate measurement of the maximum flow rate of regurgitating blood and the maximum velocity of regurgitating blood, which are used for determining the severity of a patient's valvular heart disease.

According to the above arrangement, the following effect can be obtained.

This ultrasonic diagnosis apparatus can set an ultrasonic focus position at a proper position at which it can obtain the maximum flow velocity of regurgitant blood and the maximum flow rate of regurgitant blood without requiring the user to adjust an ultrasonic focus position. This makes it possible to accurately measure the maximum flow velocity of regurgitant blood and the maximum flow rate of regurgitant blood for the determination of the severity of a patient's valvular disease.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnosis apparatus comprising:
an ultrasonic probe;
a transmission/reception unit configured to transmit and receive an ultrasonic wave to and from an object through the ultrasonic probe;
an echo signal storage unit configured to store a plurality of echo signals obtained by the transmission/reception unit;
a reception delay addition processing unit configured to generate a plurality of reception signals corresponding to a plurality of reception focus positions by performing delay addition of the plurality of stored echo signals;
a Doppler signal generating unit configured to generate a plurality of Doppler signals corresponding to the plurality of reception focus positions from the generated reception signals; and
a focus position decision unit configured to perform selection of a Doppler signal from the plurality of generated Doppler signals based on a blood flow characteristic and decide a reception focus position corresponding to the selected Doppler signal.

2. The apparatus according to claim 1, wherein the focus position decision unit regards a blood flow velocity being maximum value as the blood flow characteristic.

3. The apparatus according to claim 2, wherein the focus position decision unit regards an absolute value of the blood flow velocity being maximum value as the blood flow characteristic.

4. The apparatus according to claim 3, wherein the focus position decision unit selects a Doppler signal including a largest value from maximum absolute values of the blood flow velocities of the plurality of Doppler signals in one heartbeat period of the object.

5. The apparatus according to claim 2, wherein the focus position decision unit selects a Doppler signal including a highest velocity of the maximum blood flow velocities of the plurality of Doppler signals in one heartbeat period of the object.

6. The apparatus according to claim 1, wherein the focus position decision unit regards a blood flow rate being maximum value as the blood flow characteristic.

7. The apparatus according to claim 6, wherein the focus position decision unit selects a Doppler signal including a largest rate from the maximum blood flow rates of the plurality of Doppler signals in one heartbeat period of the object.

8. The apparatus according to claim 1, wherein the focus position decision unit regards a regurgitant blood flow velocity being maximum value as the blood flow characteristic.

9. The apparatus according to claim 8, wherein the focus position decision unit selects a Doppler signal including a largest value of the maximum values of the regurgitant blood flow velocities of the plurality of Doppler signals in one heartbeat period of the object.

10. The apparatus according to claim 1, wherein the focus position decision unit regards a regurgitant blood flow rate being maximum value as the blood flow characteristic.

11. The apparatus according to claim 10, wherein the focus position decision unit selects a Doppler signal including a largest rate from the maximum regurgitant blood flow rates of the plurality of Doppler signals in one heartbeat period of the object.

12. The apparatus according to claim 1, further comprising system controller configured to store the plurality of reception delay patterns corresponding to the plurality of reception focus positions in an internal memory, wherein the plurality of reception focus positions are set at predetermined intervals from a valve port of the heart of the object in a direction in which a depth of field increases.

13. The apparatus according to claim 1, further comprising a display unit configured to display at least one of the plurality of reception focus positions, a plurality of Doppler signals respectively corresponding to the plurality of reception focus positions, and the selected Doppler signal.

14. An ultrasonic data acquisition method comprising:
transmitting and receiving an ultrasonic wave to and from an object through an ultrasonic probe;
generating a plurality of echo signals based on the received ultrasonic waves;
storing the plurality of generated echo signals;
generating a plurality of reception signals corresponding to a plurality of reception focus positions by performing delay addition of the plurality of stored echo signals;
generating a plurality of Doppler signals corresponding to the plurality of reception focus positions from the generated reception signals;
selecting a Doppler signal from the plurality of generated Doppler signals based on a blood flow characteristic; and
deciding a reception focus position corresponding to the selected Doppler signal.

15. The method according to claim 14, wherein in the selecting, a blood flow velocity being maximum is regarded as the blood flow characteristic.

16. The method according to claim 15, wherein in the selecting, an absolute value of the blood flow velocity being maximum is regarded as the blood flow characteristic.

17. The method according to claim 16, wherein in the selecting, a largest value of maximum absolute values of the blood flow velocities of the plurality of Doppler signals in one heartbeat period of the object is regarded as the blood flow characteristic.

18. The method according to claim 15, wherein in the selecting, a highest velocity of the maximum blood flow velocities of the plurality of Doppler signals in one heartbeat period of the object is regarded as the blood flow characteristic.

19. The method according to claim 14, wherein in selecting, a blood flow rate being maximum is regarded as the blood flow characteristic.

20. The method according to claim 19, wherein in the selecting, a largest rate of the maximum blood flow rates of the plurality of Doppler signals in one heartbeat period of the object is regarded as the blood flow characteristic.

21. The method according to claim 14, wherein in the selecting, a regurgitant blood flow velocity being maximum is regarded as the blood flow characteristic.

22. The method according to claim 21, wherein in the selecting, a largest value of the maximum values of the regurgitant blood flow velocities of the plurality of Doppler signals in one heartbeat period of the object is regarded as the blood flow characteristic.

23. The method according to claim 14, wherein in the selecting, a regurgitant blood flow rate being maximum is regarded as the blood flow characteristic.

24. The method according to claim 23, wherein in the selecting, a largest rate of the maximum regurgitant blood flow rates of the plurality of Doppler signals in one heartbeat period of the object is regarded as the blood flow characteristic.

25. The method according to claim 14, further comprising setting the plurality of reception focus positions at predetermined intervals from a valve port of the heart of the object in a direction in which a depth of field increases.

26. The method according to claim 14, further comprising displaying at least one of the plurality of reception focus positions, a plurality of Doppler signals respectively corresponding to the plurality of reception focus positions, and the selected Doppler signal.
